(19) United States
(12) Patent Application Publication
(10) Pub. No.: US 2006/0257929 A1
(43) Pub. Date: Nov. 16, 2006

(54) Method for the rapid taxonomic identification of pathogenic microorganisms and their toxic proteins

(75) Inventors: Linda S. Powers, Logan, UT (US); Walther R. Ellis JR., Logan, UT (US); Christopher R. Lloyd, North Logan, UT (US)

Correspondence Address: K. S. Cornaby, Suite 1500, 170 South Main Street, Salt Lake City, UT, 84101, US

(73) Assignee: MicroBioSystems, Limited Partnership

(21) Appl. No.: 11/273,356

(22) Filed: Nov. 14, 2005

Related U.S. Application Data
(63) Continuation-in-part of application No. 10/706,543, filed on Nov. 12, 2003, now abandoned.

(51) Int. Cl. G01N 33/53

(52) U.S. Cl. 435/7.1

(57) ABSTRACT

The present invention describes a method for the rapid binding of pathogenic microorganisms and their toxic proteins with ligands that have been covalently tethered at some distance from the surface of a substrate. Ligands directed to microbes are covalently attached to the substrate surface by tethers that are between 35 Å and 50 Å in length for optimal binding efficacy. Ligands directed to capture and concentrate proteinaceous materials are covalently attached to the substrate surface by tethers that are between 35 Å and 50 Å in length for optimum assay kinetics. The ligands described herein include heme compounds, siderophores, polysaccharides, and peptides specific for toxic proteins, outer membrane proteins and conjugated lipids. Non-binding components of the solution to be analyzed are separated from the bound fraction and binding is confirmed by detection of the analyte via microscopy, fluorescence, epifluorescence, luminescence, phosphorescence, radioactivity, or optical absorbance. By patterning numerous ligands in an array on a substrate surface it is possible to taxonomically identify the microorganism by analysis of the binding pattern of the sample to the array.

### CROSS-REFERENCE TO RELATED APPLICATIONS

This application claims priority from U.S. patent application Ser. No. 10/706,543, filed 12 Nov. 2003, under the requirements of 35 U.S.C. 120, the entire contents of which are incorporated herein by reference.

### FIELD OF THE INVENTION

The present invention relates to a method for the taxonomic identification of pathogenic microorganisms and the detection of their proteinaceous toxins and other proteins of diagnostic utility.

### BACKGROUND OF THE INVENTION

Pathogenic microorganisms, particularly pathogenic bacteria which either occur naturally or which have acquired virulence factors, are responsible for many diseases which plague mankind. Many of these bacteria have been proposed as biowarfare agents. In addition, there is also the risk and likelihood that nonpathogenic microbes could also be used as pathogens after genetic manipulation (e.g., Escherichia coli harboring the cholera toxin).

Typical pathogenic bacteria include those responsible for botulism, bubonic plague, cholera, diphtheria, dysentery, leprosy, meningitis, scarlet fever, syphilis and tuberculosis, to mention a few. During the last several decades, the public perception has been one of near indifference in industrialized nations, principally because of successes that have been achieved in combating these diseases using antibiotic therapy. However, bacteria are becoming alarmingly resistant to antibiotics. In addition, there have been recent revelations of new roles that bacteria perform in human diseases such as Helicobacter pylori as the causative agent of peptic ulcers, Burkholderia cepacia as a new pulmonary pathogen and Chlamydia pneumoniae as a possible trigger of coronary heart disease. Apart from those pathogens, various socioeconomic changes are similarly contributing to the worldwide rise in food-borne infections by bacteria such as Escherichia coli, Salmonella spp., Vibrio spp., and Campylobacter jejuni.

Potential infections are also important considerations in battlefield medicine and for bioaerosol monitoring and rapid diagnostics for homeland security applications. A number of bacterial pathogens, including Bacillis anthracis and Yersinia pestis and their exotoxins, have been used as weapons. And there is always the risk that nonpathogenic microbes can be engineered to be pathogenic and employed as biowarfare agents.

Pathogenic microorganisms are also of concern to the livestock and poultry industries as well as in wildlife management. For example, Brucella abortus causes the spontaneous abortion of calves in cattle. Water supplies contaminated with exotoxin-producing microorganisms have been implicated in the deaths of bird, fish and mammal populations. More recently, mad cow disease has been traced to the oral transmission of a proteinaceous particle that is not retained by filters (classified as a prion). Thus, there is clearly a need for rapid and inexpensive techniques to conduct field assays for toxic proteins and pathogenic microorganisms that plague animals as well as humans.

As a general proposition, bacterial contamination can be detected by ordinary light microscopy. This technique, however, is only of limited taxonomic value. The investigation and quantitation of areas greater than microns in size are difficult and time consuming. Many commercially available systems rely on the growth of cultures of bacteria to obtain sufficiently large samples (outgrowth) for the subsequent application of differential metabolic tests for species (genus) identification. However, techniques requiring bacterial outgrowth may fail to detect viable but nonculturable cells. To the contrary, the growth media employed may favor the growth of bacteria with specific phenotypes.

More sensitive and more rapid typing schemes are described in “Strategies to Accelerate the Applicability of Gene Amplification Protocols for Pathogen Detection in Meat and Meat Products” by S. Pillai and S. C. Ricke (Crit. Rev. Microbiol. 21(4), 239-261 (1995)) and “Molecular Approaches for Environmental Monitoring of Microorganisms” by R. M. Atlas, G. Sayler, R. S. Burlage and A. K. Bej (Biotechniques 12(5), 706-717 (1992)). Those techniques employ the polymerase chain reaction (PCR) for amplification of bacterial DNA or RNA, followed by nucleic acid sequencing to detect the presence of a particular bacterial species. Such general amplification and sequencing techniques require technical expertise and are not easily adaptable outside of specialized laboratory conditions. PCR-based techniques utilize the inference of microbial presence since these techniques provide only a positive analysis whenever an intact target nucleic acid sequence, not necessarily a microbe, is detected. PCR is also unable to detect the presence of toxic microbial proteins or other proteinaceous materials. Moreover, the detection of specific microorganisms in environmental samples is made difficult by the presence of materials that interfere with the effectual amplification of target DNA in ‘dirty’ samples. In many circumstances, extensive sample preparation steps are necessary to isolate the nucleic acid sequences from interfering materials, thus increasing the cost and time required to determine the presence of microbial nucleic acid sequences in a sample.

Mass spectral analysis of volatile cell components (e.g., fatty acids) after sample lysis or pyrolysis has been used for the detection of bacteria and viruses. One description of the methods used to detect microorganisms with this method can be found in “Characterization of Microorganisms and Biomarker Development from Global ESI-MS/MS Analyses of Cell Lysates” by F. Xiang, G. A. Anderson, T. D. Veenstra, M. S. Lipton and R. D. Smith (Anal. Chem. 72 (11), 2475-2481 (2000)). Unfortunately, identification of the analyte is unreliable as the compositions of a microbe's volatile components change depending upon different environmental growth conditions.

Another approach utilizes immunochemical capture as described in “The Use of Immunological Methods to Detect and Identify Bacteria in the Environment” by M. Schlotter, B. Assmus and A. Hartmann (Biotech. Adv. 13, 75-80 (1995)), followed by optical detection of the captured cells. The most popular immunoassay method, enzyme-linked immunosorbent assay (ELISA), has a detection limit of several hundred cells. This is well below the ID₅₀ of extremely infectious bacteria such as Shigella flexneri. Piezoelectric detection techniques, such as those described by “Development of a Piezoelectric Immunosensor for the Detection of Salmonella typhimurium” by E. Prusak-Sochaczewski and J. H. T. Luong (Enzyme Microb. Technol. 12: 173-177 (1990)) are even less sensitive having a detection limitation of about 5×10⁵ cells. A recent report entitled “Biosensor Based on Force Microscope Technology” by D. R. Baselt, G. U. Lee and R. J. Colton (Biosens. & Bioelectron. 13, 731-739 (1998)) describes the use of an atomic force microscope (AFM) to detect immunocaptured cells; this method has little utility outside a laboratory setting and when the sample volumes are large. Immunoassays are also presently used in the trace analysis of peptides and proteins.

Moreover, the prior art has made extensive use of immobilized antibodies in peptide/protein/microorganism capture. Those techniques likewise involve significant problems because the antibodies employed are very sensitive to variations in pH, ionic strength and temperature. Antibodies are susceptible to degradation by a host of proteolytic enzymes in “dirty” samples. In addition, the density of antibody molecules supported on surfaces (e.g., microwell plates or magnetic beads) is not as high as is frequently necessary. A good summary of the state of the art, still up-to-date, is “Microbial Detection” by N. Hobson, I. Tothill and A. Turner (Biosens. & Bioelectron. 11, 455-477 (1996)). Immunoassays for microbial and proteinaceous targets generally call for relatively long incubation steps to achieve binding equilibrium between immobilized antibody and antigen-containing solution; this is particularly true for assays designed to detect low levels of analyte. The Methods in Molecular Biology series publication “ELISA: Theory and Practice” by J. Crowther ([1995] Humana Press, New Jersey) directs immunoassay developers to use incubation conditions varying between one to three hours for stationary incubations (p. 74) and of at least one hour for individual incubation steps in sandwich assays (p. 164-165). (Some immunoassays call for overnight incubations with the target solution.) The relatively long incubation steps utilized in many immunoassays renders them less effective for rapid detection of low levels of microbial analytes.

Medical and military considerations call for better toxin and pathogen detection technologies. Real-time assessment of battlefield contamination by a remote sensing unit is necessary to permit and facilitate rapid diagnosis for administration of appropriate counter-measures. A microbe/toxic protein sensor useful in such situation requires the ability to globally discriminate between pathogens and non-pathogens. In addition, such techniques require high sensitivity when less than 100 cells are present and analysis that can be completed rapidly in the field (optimally in less than 15 minutes). Such techniques should be able to recognize pathogens and provide some assessment of strain virulence or toxigenicity.

To date, common approaches used for the identification of pathogenic microorganisms and their proteinaceous toxins have employed immunological methodologies. Immunological methods suffer from the sensitivity of antibodies toward pH, ionic strength, and temperature; the antibodies themselves are subject to proteolysis and require careful storage conditions. To overcome these problems the present invention describes the capture of microorganisms and their proteinaceous toxins using non-antibody based ligands that have been covalently tethered to substrate surfaces at optimal lengths for binding efficiency and assay speed. It is accordingly an object of the present invention to provide a method for taxonomically evaluating microbes and proteins that overcome the foregoing disadvantages of technologies that depend upon antibodies.

It is a more specific object of the invention to provide a method for taxonomically evaluating microbes and proteins that has the capability of discriminating between specific microbial species, pathogens and nonpathogens, and can be likewise used to identify microbial proteins of diagnostic utility.

### SUMMARY OF THE INVENTION

The present invention demonstrates the ability of heme compounds, siderophores, polysaccharides and peptides to bind to pathogenic microorganisms and their proteinaceous toxins; taxonomic identification of a microorganism is attained through analysis of the number and kind of ligands to which it binds. The development of this method was done to overcome the aforementioned limitations of antibody-based technologies. The concept of the present invention resides in a method for the taxonomic identification of microorganisms in which microbes are captured through the binding of microbial receptors to specific surface-tethered ligands. A microorganism-containing sample is contacted by the ligand, with the ligand being either tethered to a surface or conjugated to a marker. The target microbe (bacteria, virus, fungi, protozoa, rickettsiae, or other cell) or proteinaceous material (toxin, prion or other protein of diagnostic utility) is then separated from the non-binding sample components and unbound ligand as by washing, magnetic separation, chromatography or the like. Finally, the sample is interrogated by an appropriate method to determine if the ligand has been bound to the target by detecting signals endogenous to the target or marker.

Electromagnetic radiation is one method used to detect the presence of metabolites characteristic of living microbes, e.g., reduced pyridine nucleotides or other fluorescent metabolites, other biomolecules, e.g., notably tryptophan or tyrosine in proteins, or incorporated dyes for the detection of the presence of the captured microorganisms and/or toxins in accordance with the practice of the invention. For example, if the ligand contains a fluorescent dye, the sample will fluoresce after washing, since the ligand is bound to the cells and the excess is washed away. Other markers, including luminescent, phosphorescent, radioactive and/or colorometric compounds, can be conjugated to the ligand and used to identify a microbe and/or proteinaceous toxin in a similar manner.

Methods used to detect the presence of captured microorganisms or toxic proteins are described in U.S. Pat. Nos. 5,760,406; 5,968,766 and 6,750,006 where electromagnetic radiation is directed, for example, onto the surface of a ligand-conjugated substrate that has been treated with an analyte-containing solution as outlined above. This detection method could be used to determine if binding of an analyte has occurred. Other detection methods, appropriate for the specific kind of marker conjugated to the ligand, can also be employed to determine if the ligand has been specifically bound to a microorganism or toxic protein. An example mentioned previously uses a fluorescent dye conjugated to a ligand coupled to detection of a microbe via fluorescence characteristic of the dye after (1) contact between the microbe and ligand and (2) washing away excess dye-conjugated ligand. It is important to note that if optical methods are used to detect the captured microbe or protein the tether should not be photocleavable (i.e., it should be photostable).

Thus, the method of the present invention does not depend on classical antigen-antibody recognition. On the contrary, the concepts of the present invention make use of relatively inexpensive reagents in the capture of microorganisms and microbial proteins contained in the sample.

In one embodiment of the invention, sensor chips (or beads) substrates are employed. These sensor chip or bead substrates should be formed from a suitable support material such as glass or plastic (e.g., poly(propylene) or poly(vinyl acetate)) that will be compatible with both the chemistries used to conjugate the linker and ligand to the surface and the detection method employed. The sensor chip is formed of a patterned array defining a plurality of sections on the surface of the sensor chip, and each section has bonded thereto a different ligand capable of molecularly recognizing a specific microbial protein or microbial receptor, and hence the microbe itself. Microbial receptors would include, for example, proteins residing in the outer membrane of the microbial cell, pilus or flagellum, which is exposed to the aqueous environment surrounding the cell. The ligand for pathogen/protein capture bonded to the surface of the sensor chip can and should be varied. In general, such ligands may be characterized as heme compounds, siderophores, polysaccharides and anti-adhesion peptides capable of capturing a wide variety of microorganisms and toxic proteins. Chemical compounds similar to the substrates of membrane-associated enzyme substrates but which cannot undergo the associated reaction may also be employed as ligands. These ligands can thus be immobilized or bonded to the surface of the sensor chip through an appropriately sized cross-linker also having the capability of reacting with the ligands, whereby the coupling agent establishes a chemical tether between the surface of the sensor chip and the ligand capable of reaction with a variety of different microorganisms and proteins. The size (length) of the tether is chosen to optimize binding efficiency of the target and to provide the most advantageous assay times. The sensor chips and arrays (1) are exposed to a solution containing microorganisms or toxic proteins, (2) the non-binding constituents of the solution are removed, (3) followed by interrogation of the ligand-tethered surfaces to detect analyte binding. Analysis of the type or pattern of ligand-tethered surfaces found to have captured the microorganism(s), or microbial proteins not contained within intact microbial cells, can be used to taxonomically identify a microorganism or its toxic protein.

Thus, the present invention can be used rapidly to identify microorganisms without the need for growing a culture of the microorganism and then microscopically examining the culture thus produced. Likewise, low levels of toxic microbial proteins or other proteinaceous material of diagnostic utility can similarly be identified. It is also unnecessary to employ enzymes or antibodies in the capture of microbial metabolites as is often used in the prior art. These, and other objects, features and advantages of the present invention will become apparent upon review of the following detailed descriptions of the disclosed embodiments and the appended claims.

### BRIEF DESCRIPTION OF THE FIGURES

FIG. 1 shows the capture of Salmonella typhimurium from a solution on a glass microscope slide coated with tethered heme. Detection of this pathogenic bacterium, which is indicated by F/R (fluorescence/reflectance ratio), was accomplished according to the method and apparatus outlined in U.S. Pat. No. 5,968,766 to Powers.

FIG. 2 shows the capture of Enterobactor aerogenes diluted in various concentrations of Bacillus globigii on a glass microscope slide coated with tethered heme. Detection of this pathogenic bacterium, which is indicated by F/R (fluorescence/reflectance ratio), was accomplished according to the method and apparatus outlined in U.S. Pat. No. 5,968,766 to Powers.

FIG. 3 shows the effect of the length of the tether between a surface and a ligand (iron-containing deferoxamine) on the binding of a microorganism (Klebsiella pneumoniae). At shorter tether lengths (<10 Å) binding is superior to that observed at medium lengths (ca. 20 Å) and optimizes at lengths at around 40 Å; longer tether lengths affect affinity little. Detection of this pathogenic bacterium was accomplished via the intrinsic fluorescence from the bacteria.

FIG. 4 shows the kinetic binding curves of fluorescent dye-conjugated Staphylococcal enterotoxin B by a peptide ligand tethered at around 40 Å (solid square, ▪), a peptide ligand tethered at around 30 Å (open circle, ∘) and by a surface adsorbed antibody (solid triangle, ▴). Detection of the bound toxin was accomplished via the fluorescence of the conjugated dye. The capture of the dye-conjugated toxin from solution was biphasic, with the slower rate constant independent on tether length or ligand and the faster rate constant of the tethered peptide ligands dependent on the tether length.

FIG. 5 shows the effect of the length of the tether between a surface and a ligand (peptide specific for Staphylococcus enterotoxin B) on the faster binding rate constant for the capture of dye-conjugated Staphylococcus enterotoxin B from solution.

FIG. 6 shows the effect of washing on the retention of bound Staphylococcus aureus that has been captured from solution to a surface with a peptide ligand tethered 41 Å from the surface (solid square, ▪) and by a surface adsorbed antibody (solid triangle, ▴).

### DETAILED DESCRIPTION OF THE INVENTION

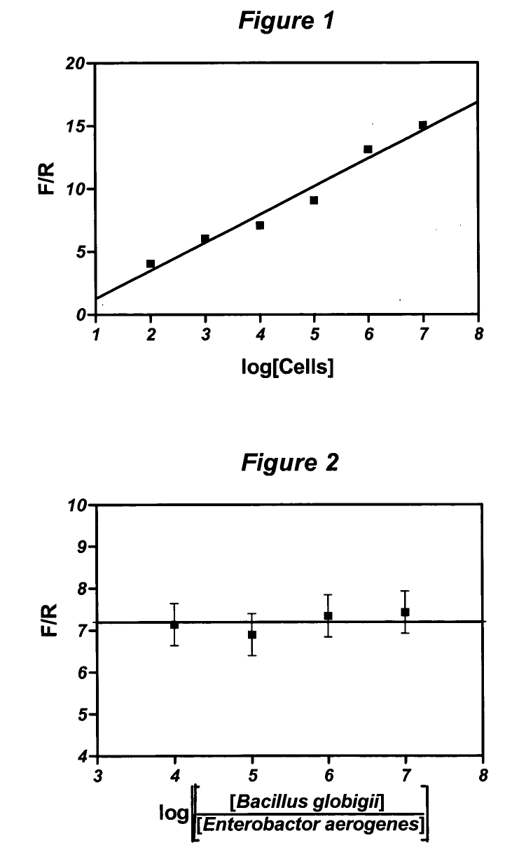

The capture of a pathogenic bacterium (Salmonella typhimurium) with tethered heme, as outlined in the present invention, is shown in FIG. 1. The method and apparatus outlined in U.S. Pat. No. 5,968,766 was employed for the detection of the captured bacteria. Although numerous compatible bacterial detection methods could have been employed, this method was used due to its ability to detect such small numbers of bacteria on the slide. Inspection of the figure shows that the detection limit (<100 cells) of the captured microorganism using a tethered heme ligand is lower than that observed using immunological methods (ca. 400 cells under optimal conditions). Binding between the microorganism and the heme ligand is not as sensitive to pH, ionic strength and temperature as is binding to an antibody. The heme ligand is also less expensive, requires less careful storage and is not susceptible to proteolysis as are antibodies.

FIG. 2 shows the tethered heme capture of a pathogenic bacterium (Enterobactor aerogenes) that has been diluted to the same concentration in solutions of a nonpathogen (Bacillus globigii). This figure shows that the tethered heme-coated slide is able to effectively capture the pathogenic bacteria from a solution even when the nonpathogen to pathogen ratio is 10⁷:1. Detection of the captured bacteria was accomplished with the apparatus outlined in U.S. Pat. No. 5,968,766 to Powers.

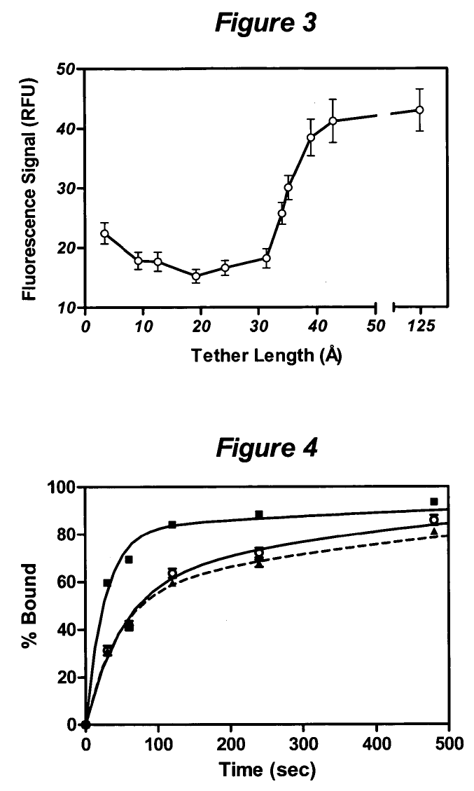

In the preferred practice of the invention, the linker should be of sufficient length to present the ligand at the optimal distance (around the distance of 40 Angstroms) from the surface of the chip. This observation is based on our determination that shorter distances results in decreased bacterial cell capture efficiency. The effect of the tether length is illustrated in FIG. 3. This figure shows the effect of the length of the tether between a surface and a ligand (iron-containing deferoxamine) on the binding of a microorganism (Klebsiella pneumoniae). (Detection of this pathogenic bacterium was accomplished via the intrinsic fluorescence from the bacteria.) At shorter tether lengths (<10 Å) binding is superior to that observed at medium lengths (ca. 20 Å) and optimizes at lengths at around 40 Å; longer tether lengths affect affinity little. Increasing tether lengths beyond 50 Å has, in some cases, been shown to increase non-specific binding of non-target analytes to the tether.

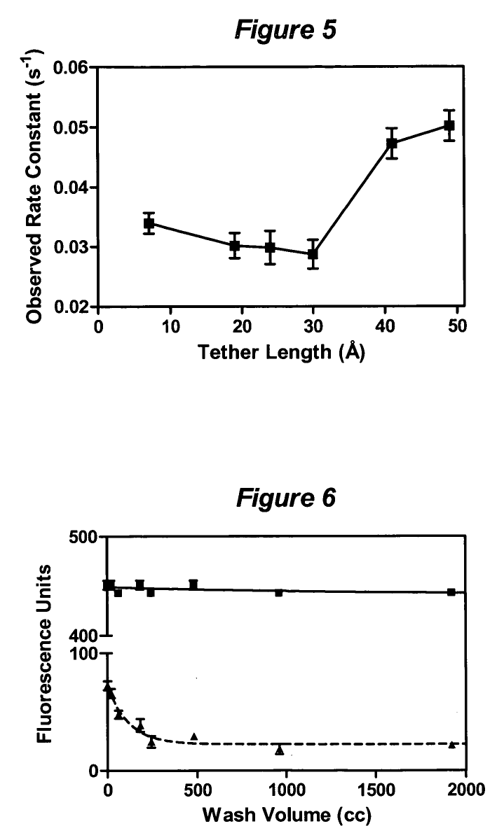

FIG. 4 shows the kinetic binding curves of fluorescent dye-conjugated Staphylococcal enterotoxin B by a peptide ligand (WHKAPRGGGGC) tethered at around 40 Å (solid square, ▪), the same peptide ligand tethered at around 30 Å (open circle, ∘) and by a surface adsorbed antibody (solid triangle, ▴). The dye-conjugate was prepared by exposing an excess of Staphylococcus enterotoxin B to sulfosuccinimidyl-7-amino-4-methylcoumarin-3-acetic acid. Detection of the bound toxin was accomplished via the fluorescence of the conjugated dye. In this example of the capture of a proteinaceous toxin, the binding of the dye-conjugated toxin from solution was biphasic, with the slower observed rate constant independent of tether length or ligand (ca. 10⁻³ sec⁻¹ at 30° C.). The faster observed rate constant for the tethered peptide ligands exhibits a dependence on the tether length, with faster binding kinetics realized with tether lengths greater than 30 Å (optimally between 35 Å and 50 Å), as shown in FIG. 5. It will be appreciated by those skilled in the art (and by inspection of FIG. 4) that the ligands tethered to the surface at lengths between 35 Å and 50 Å capture the protein analyte from solution ten times faster than antibody coated surfaces. (Five half-lives of the fast phase of the peptide ligand tethered at 41 Å occur in less than two minutes time.)

FIG. 6 shows the effect of washing on the retention of bound Staphylococcus aureus that has been captured from solution to a surface with a peptide ligand tethered 41 Å from the surface (solid square, ▪) and by a surface adsorbed antibody (solid triangle, ▴). In this experiment, Staphylococcus aureus (suspended in a pH 7.5 phosphate buffered saline) was exposed to a peptide ligand specific for Protein A (GHHKHHGGGC) that had been pre-treated with (N-[6-7-amino-4-methylcoumarin-3-acetamido]hexyl)-3′-[2′-pyridyldithio]propionamide to couple the fluorescent dye to the cysteine of the peptide ligand. Fifty microliter droplets of the dye-conjugated, ligand-bound bacteria solution were placed on surfaces containing either a tethered peptide ligand specific for Protein A or an antibody to Protein A and allowed to stand for 20 minutes. (Protein A is a surface protein of S. aureus.) The surfaces were washed with a minimal amount of distilled water (ca. 5 mL) and the presence and relative amount of the captured and labeled S. aureus cells was determined by the method described in U.S. Pat. No. 6,750,006. Each of the surfaces were washed with increasing amounts of distilled water, and the relative amount of bound bacteria was determined via the sum of the fluorescence signal from the dye-conjugated peptide and the reduced pyridine nucleotides contained in the bacterium itself. Two things can be noticed from examination of FIG. 6: first, the tethered ligands captured more bacteria than the antibody-coated surface; and second, washing notably decreased the amount of antibody-captured S. aureus. Between 10³ and 10⁴ tethered ligands will fit on the surface in the same area covered by the diameter of the Stokes radius of an antibody molecule. Since there were more tethered ligands on the surface than antibody molecules, the peptide-conjugated surface had a larger binding capacity (as seen by the larger initial signal). Furthermore, since each S. aureus bacterium contains multiple copies of Protein A on its surface, each microbe could be bound by multiple ligand-target interactions. This makes the covalently tethered ligand-target analyte interaction harder to dissociate, resulting in a more stable overall interaction and capture.

The observation that the binding of analytes to tethered ligands is biphasic has important implications for assay development using the method described by this invention. Since the use of optimal tether lengths decreases the contact time between the fluid containing the analyte and the substrate bearing the tethered ligands, sample exposure times to the binding surfaces can be reduced significantly. Thus, in the practice of this invention, the tethers are used to both maximize binding efficiency (increased binding constants) and decrease assay times (increased apparent binding rate constants). In the example shown in FIG. 4, the same amount of toxin is captured from solution by the tethered peptide ligand surface in fifty seconds as is captured by the surface adsorbed antibody substrate in five hundred seconds. The observation that the binding is biphasic, and that the effect of the tether length on analyte capture is observed for the fast phase of that binding, means that the majority of the benefits of this effect are realized within five half-lives of the fast phase. This benefit can be realized in the practice of the invention by exposing the tethered ligand surface to the fluid containing the analyte for three to five half-lives of the fast binding phase, instead of the one to eight hour incubations used in antibody-based methods.

It is important to note that using a tether of the appropriate length not only decreases the required exposure time of the analyte solution to the tethered ligand substrate, but that bound analytes exhibit greater retention. Similar to the situation of the bound Staphylococcus aureus shown in FIG. 6, tethered-ligand captured Staphylococcus enterotoxin B exhibits greater retention than that immobilized by surface-adsorbed antibodies. This is an important feature in the practice of this invention for the step in which the bound analyte is separated from the non-binding material in the analyte solution. Since bound material is not easily removed by either washing or subsequent exposure to further analyte solution, tethered ligand substrate surfaces can be used to concentrate analytes from solution. This feature can be utilized to decrease assay detection limits by improving sampling methods, or as a stationary phase for chromatographic separation.

Another important feature of this invention is that since assay times can be reduced, and since the materials used to produce the tethered ligands are comparatively inexpensive, the practice of this invention is especially appropriate to use in situations where rapid assays are needed. Examples would be a point-of-care assay for a sexually transmitted disease (e.g., gonorrhea), for an infectious disease transmitted by aerosols (e.g., tuberculosis), or for other situations where urgent treatment would decrease public health risks. The method would also be appropriate for samples that are ‘dirty’ (i.e., contain a relatively high amount of non-binding components) or for where sampling needs to occur from relatively fast-flowing fluids.

In one embodiment of the present invention, a sample containing an unknown analyte microorganism or protein toxin is first contacted by the ligand. The ligand can be tethered to a surface of either a chip or bead. Binding efficiency is dependent upon the length of the tether. Microbes are found to bind most efficiently to ligands that are around forty Å long. Ligands directed to microbes are covalently attached to the substrate surface by tethers that are between 35 Å and 50 Å in length for optimal binding efficacy. Ligands directed to capture and concentrate proteinaceous materials are covalently attached to the substrate surface by tethers that are between 35 Å and 50 Å in length for optimum assay kinetics. The analyte is then physically separated from the non-binding sample. Analytes captured by ligands tethered to a surface can be separated from non-binding components of the sample by simply washing the surface of the chip or bead. The surface of the substrate is then interrogated to determine if binding of the analyte to the ligand has occurred. The detection of bound microbes on the substrate surface can be made with: microscopy, intrinsic fluorescence, conjugate dye fluorescence, radioactivity, luminescence, phosphorescence, and/or optical absorbance. Identification of the microbe or protein is determined by the identity of the ligand. It is important to note that the tether should be photostable or not otherwise chemically labile in the solution used to wash the ligand-tethered surface.

In another embodiment of the invention, taxonomic identification is made by identification of proteins that are found in the cytosol (or interior) of the microorganism. To expose the target analyte (proteinaceous material) to a substrate exhibiting tethered ligands it is necessary to rupture the microorganism resulting in the spilling of the analyte into a solution. This rupture can occur through numerous chemical treatments (resulting in the destabilization and degradation of membranes), enzymatic treatments (including, but not limited to treatment with holins or other enzymes), physical treatments (including, but not limited to plasma discharge, freeze-thaw cycling, sonication, shearing and the like), or infection (bacteriophage infection). The solution that now contains solublized analyte is now exposed to the substrate-tethered ligands, separated from the solution, and the substrate is interrogated to determine if binding to the analyte has occurred, whereby presence of the analyte is provided through detection of binding between the analyte and the analyte-specific ligand.

In one embodiment of the invention, a sample containing an unknown analyte microorganism or protein is first contacted with the sensor chip. The sensor chip is formed of a substrate, such as glass, having a series of sections on the surface thereof. Each section has a different ligand bonded thereto, so as to be capable of binding to specific analytes. The ligands are capable of binding to the analyte for capture, and the presence of the captured analyte is detected using a fluorescence detection system, for example, disclosed and claimed in U.S. Pat. Nos. 5,760,406 and 5,968,766 and via the intrinsic fluorescence of the proteinaceous toxins. Thus, the ligand of each of the sections of the sensor chip has the capability of capturing a specific microbial cell or microbial protein. The used chip can be saved and used to grow out the captured microorganisms as well.

In one embodiment of the present invention, a sample fluid (liquid solution, suspension or aerosol containing suspended biological analyte) containing an unknown analyte microorganism or protein is first contacted by the tethered ligand conjugated surface. The ligand can be tethered to the substrate surface of either a chip or bead by tethers that are between 35 Å and 50 Å in length for optimal binding efficacy and assay speed. The analyte is then physically separated from the non-binding sample using methods known to those skilled in the art. An example of a separation method is to use chromatography, where the substrate serves as the stationary phase, and the fluid (or alternately and preferably wash solution) serves as the mobile phase that separates the non-bound components of the fluid from the substrate surface. Since prompter ‘fast phase’ analyte binding kinetics are observed for tethered ligands whose tethers are the appropriate length as described above, this invention is uniquely capable of capturing a greater portion of target analytes from fast flowing applications. The surface of the substrate is then interrogated to determine if binding of the analyte to the ligand has occurred. Alternately, the analyte can be physically separated from the non-binding sample using magnetic separation, wherein the ligand is conjugated to a magnetic particle and the separation of the bound analyte from the non-binding components of the analyte solution is accomplished by magnetic separation with the ligand being tethered to the magnetic particle.

In an alternate embodiment of the invention, a sample containing an unknown analyte (microorganism, proteinaceous toxin or other protein) is first contacted with a ligand conjugated to a marker, including, but not limited to, a fluorescent dye. The non-binding sample components and excess ligand are separated from the ligand-bound analyte; this separation can be accomplished by centrifugation (for cells), magnetic sedimentation or chromatography for proteins. The detection of binding between the analyte and ligand, and thus taxonomic identification of the analyte, is accomplished by detection of the marker (e.g., fluorescence of the dye-conjugate in the example above).

In another embodiment of the invention, a sample containing an unknown analyte, such as, a microorganism or protein is first contacted with a ligand tethered to a substrate surface with a linker of appropriate length, as noted above. Physical separation and washing remove non-binding components of the solution. As will be appreciated by those skilled in the art, the captured microorganism or protein can be treated with a reactive marker, provided the marker does not react with either the substrate surface or the ligands. Detection of the marker on the area of the surface associated with the ligand(s) that have been exposed to the analyte indicates the presence of a specific analyte.

In yet another embodiment of the invention, a sample containing an unknown analyte microorganism or protein is contacted with a substrate surface conjugated with tethered ligands. The substrate surface should not be soluble to the solution containing the unknown analytes or any wash solution. The substrate should be a material suitable to allow the sample solution to flow over and through the substrate, thus capturing and concentrating the analyte. The tethered ligands conjugated to the surface must be capable of binding to the analyte, and the presence of the captured and now concentrated analyte can be detected using a number of optical methods. Concentration of the biological analyte from a complex mixture is especially useful for contaminated water samples, medical samples, veterinary samples, aerosol samples, food product slurries, food ingredient slurries and soil slurries.

In a preferred embodiment of the invention, the ligands used in the present invention may be taken from the group comprised of heme compounds, siderophores, polysaccharides (including oligosaccharides) and peptides.

As is also well known to those skilled in the art, animal pathogens generally possess heme uptake capability, and thus heme compounds can be used to capture a number of pathogenic species. In addition to heme compounds, other ligands in the form of high-affinity iron chelators, generally referred to as siderophores, can also be used to capture many strains of pathogenic bacteria. Included among such siderophores are alcaligin, mycobactins, pyochelin, staphyloferrin, vibriobactins and yersiniabactins.

As is also well known to those skilled in the art and as mentioned above, discrimination of animal pathogens by binding to heme compounds and siderophores that have been labeled with markers is also possible. An example would include the incubation of bacteria-containing solutions with a siderophore or heme compound that has been conjugated with a fluorescent, luminescent, phosphorescent, chemiluminescent, or radioactive compound. After washing the cells, detection of animal pathogens can be made by standard fluorescence, colorimetric or radiation detection techniques. The binding of animal pathogens to heme compounds and siderophores that are tethered to a support can also be exploited to separate these microbes from environmental samples, e.g., water, for the purpose of concentration and/or purification.

In addition to heme compounds and siderophores, eukaryotic surface epitopes (peptides or carbohydrates), which are recognized by microbial cell receptors, can likewise be used as ligands in the practice of the present invention. These ligands include naturally occurring oligosaccharides and polysaccharides as well as those available by chemical synthesis. Other oligosaccharides and their affinity to pathogens from various microorganisms are described by K. A. Karlsson “Microbial Recognition of Target Cell Glycoconjugates” (Structural Biology 5:622-635 (1995)).

The characteristics of a number of pathogenic bacterial organisms, including the disease caused by each species and their binding characteristics with siderophores, oligosaccharides and heme compounds are set forth in Table I. These characteristics can be used in the capture and identification of such species.

Peptide ligands can typically be identified by affinity panning of libraries of oligopeptides and then synthesized chemically. Siderophore ligands can be produced by chemical synthesis or isolation from spent microbial culture media. Oligosaccharide ligands can be produced by chemical synthesis or isolated from eukaryotic tissue. Heme compounds can be produced typically by chemical synthesis using protoporphyrin IX as a starting reagent.

TABLE I
Bacterial Characteristics for Siderophore, Oligosaccharide and Hemin Binding

| Bacterial | Disease | Siderophore | Oligosaccharide | Hemin | Exotoxin |
|---|---|---|---|---|---|
| Species | Caused | Binding? | Binding? | Binding? | Produced? |
| Bacillus | Anthrax | Anthrabactin | a pulmonary | unknown | anthrax toxin |
| anthracis | | | oligosaccharide | | anthralysin O |
| Bordetella | Whooping | Alcaligin, | N-acetyl- | Yes | pertussis toxin |
| pertusis | cough | others | glucosamine | | |
| Clostridium | Botulism | unknown | unknown | Yes | botulinum |
| botulinum | | | | | toxin A |
| Clostridium | Gas gangrene | unknown | unknown | unknown | α-toxin, |
| perfringens | | | | | perfringolysin O |
| Clostridium tetani | Tetnus | unknown | unknown | unknown | tetanus toxin |
| Corynebacterium | Diphtheria | Aerobactin | unknown | unknown | diphtheria |
| diphthariae | | | | | toxin |
| Escherichia coli | Numerous | many | Globobiose, | Yes | Shiga-like |
| 0157:H7 | infections | | others | | toxin, others |
| Haemophilus | Meningitis | Enterobactin | GalNAcβ(1- | Yes | unknown |
| influenzae | | | 4)Gal, others | | |
| Helicobacter | Gastric ulcers | unknown | a mucosal | Yes | vacuolating |
| pylori | | | oligosaccharide | | cytotoxin A |
| Klebsiella | Numerous | many | GalNAcβ(1- | Yes | unknown |
| pneumoniae | infections | | 4)Gal, others | | |
| Mycobacterium | Tuberculosis | Mycobactin T | unknown | unknown | unknown |
| tuberculosis | | | | | |
| Neisseria | Meningitis | many | unknown | Yes | unknown |
| meningitidis | | | | | |
| Pseudomonas | Numerous | Pyochelin, | Asialo G_{M1}, | Yes | exotoxin A, |
| aeruginosa | infections | others | others | | elastase, others |
| Salmonella typhi | typhoid fever | many | unknown | Yes | Yes |
| Serratia | numerous | Aerobactin, | Yes | Yes | serralysin |
| marescens | infections | Ferrioxamine B | | | |
| Shigella | dysentery | Enterobactin, | Yes | Yes | Shiga toxin |
| dysenteriae | | Aerobactin | | | |
| Staphylococcus | numerous | Staphyloferrin, | GalNAcβ(1-4)Gal | Yes | several |
| aureus | infections | others | | | superantigens |
| Streptococcus | pneumonia, | unknown | GlcNAcetyl(1- | Yes | streptolysin O |
| pneumoniae | meningitis | | 3)Gal, others | | |
| Vibrio cholerae | cholera | Vibriobactin, | Yes | Yes | cholera toxin |
| | | others | | | |
| Yersinia pestis | bubonic | Yersiniabactin, | unknown | Yes | YopE, others |
| | plague | others | | | |

Toxins that contain at least one tryptophan, a few tyrosines or a few phenylalanines per molecule can be detected by tryptophan/tyrosine fluorescence after capture using a tethered peptide. It will be appreciated by one skilled in the art that if one uses a peptide to capture a protein, and that one wishes to detect the captured protein with intrinsic fluorescence, that it is optimal that the peptide used to capture the protein should not contain tryptophan and/or many tyrosines/phenylalanines. A variety of microbes, including algae, fungi, and bacteria, export exotoxins that are amenable to detection using this technology.

Table II contains examples of toxic, bacterial proteins that can be (1) captured using the technology described herein, and (2) ultimately detected via means of their intrinsic fluoresence. It is important to note that, for Staphylococcus aureus enterotoxin B, which represents the most unfavorable case in Table II (due to the presence of just one Trp and 22 Tyr), the following fluorescence study of the sole Trp residue has appeared: B. R. Singh, M. L. Evenson and M. S. Bergdahl “Structural Analysis of Staphylococcal Enterotoxins B and C1 Using Circular Dichroism and Fluorescence Spectroscopy” (Biochemistry 27: 8735-8741 (1988)). As is well known to those skilled in the art, detection of tryptophan/tyrosine fluorescence (normalized to the scattered excitation signal) is sufficient to indicate that spores, nonviable cells, viable vegetative bacterial or fungal cells, viruses, or a microbial toxin are present (i.e., bound to a ligand) on the surface of a sector of the sensor chip.

TABLE II
Amino Acid Counts for Selected Bacterial Toxins

| | | No. Amino | No. | No. |
|---|---|---|---|---|
| Bacterium | Toxin | Acids | Trp | Tyr |
| B. anthracis | protective antigen | 753 | 7 | 27 |
| B. anthracis | lethal factor | 770 | 5 | 35 |
| B. cereus | phospholipase C | 245 | 9 | 15 |
| B. pertussis | pertussis toxin | 952 | 11 | 50 |
| C. botulinum | toxin A | 1296 | 15 | 67 |
| C. difficile | toxin A | 2366 | 25 | 166 |
| C. perfringens | iota-toxin | 346 | 4 | 18 |
| C. tetani | tetanus toxin | 1421 | 13 | 78 |
| C. diphtheriae | diphtheria toxin | 534 | 5 | 16 |
| E. coli | alpha-hemolysin | 1023 | 3 | 38 |
| H. pylori | vacuolating cytotoxin A | 808 | 8 | 14 |
| L. monocytogenes | listeriolysin O | 523 | 7 | 23 |
| P. aeruginosa | elastase | 301 | 4 | 22 |
| S. marescens | serralysin | 470 | 7 | 19 |
| S. dysenteriae | Shiga toxin | 638 | 7 | 17 |
| S. aureus | enterotoxin B | 239 | 1 | 22 |
| S. aureus | toxic-shock toxin-1 | 194 | 3 | 9 |

Thus, as described above, a different ligand is tethered to each of the sections of the sensor chip. The sensor chip is then contacted with a sample containing unknown organisms or proteins, whereby specific ligands on the surface of the chip bind to specific analytes, selectively capturing them. The unbound analytes are then washed away with an appropriate solution (such as a phosphate-buffered saline); and the sensor chip is then subjected to an appropriate detection technique. One possible technique used to detect the presence of bacteria on the sections of the sensor chip is disclosed in U.S. Pat. Nos. 5,760,406 and 5,968,766, wherein the described apparatus utilizes electromagnetic radiation of appropriate wavelengths to excite fluorescence characteristic of the presence of bound analytes.

As is well known to those skilled in the art, if a tethered ligand used to capture an analyte is itself fluorescent then this fluorescence may change upon binding to the analyte. This change in fluorescence could be manifest as either a change in intensity or a shift of the characteristic fluorescence energy. This change in the fluorescence of the tethered ligand can be used to confirm detection of the analyte.

In the presence of the present invention, a sample containing unknown microbes can be contacted with the sensor chip, whereby one or more receptors of the bacteria react with various different ligands tethered to the various sections of the chip. Then, the fluorescence of the chip can be measured with a probe for the purpose of detecting which of the sections of the sensor chip have analytes bonded thereto. As examples, mycobacterial siderophores can be used to capture mycobacteria such as Mycobacterium tuberculosis. Helicobacter pylori can be captured using tethered N-acetylneuroaminyl-alpha-2,3-galactose. The peptide:
GADRSYLSFIHLYPELAGAGGGC
   can be tethered, by means of the terminal cysteine group to expressly capture free Staphylococcus aureus toxic-shock toxin-1. The peptide:
GHHKHHHGGGC
   can be tethered also by means of the terminal cysteine group, to specifically capture the surface-exposed protein A of Staphylococcus aureus, and hence this organism itself. The Staphylococcus aureus toxic-shock syndrome toxin-1-binding peptide was described by A. Sato, et al. in “Identification from a Phage Display Library of Peptides that Bind to Toxic Shock Syndrome Toxin-1 and that Inhibit Its Binding to Major Histocompatibility Complex (MHC) Class II Molecules” (Biochemistry 35, 10441-10447 (1996)).

As indicated above, determining the presence of a single captured microorganism or discrete microbial protein can identify some of the analytes of interest. In other cases, however, a series of two or more captured analytes of interest is indicative of the identity of a particular analyte. As an example, consider a sensor chip having an area of three sections along the horizontal axis and three sections along the vertical axis as illustrated below:

| | | |
|---|---|---|
| A1 | A2 | A3 |
| B1 | B2 | B3 |
| C1 | C2 | C3 |

In this example, the sections identified can be provided with the following ligands tethered to a specific section as set forth in the following table:

| Section Location | 3 × 3 Array Ligand |
|---|---|
| A1 | asialo G_{M1} |
| A2 | hemin |
| A3 | pyochelin |
| B1 | GalNAcβGal |
| B2 | alcaligin |
| B3 | fibronectin (peptide fragment) |
| C1 | anti-S. aureus protein A peptide |
| C2 | staphyloferrin |
| C3 | ferrioxamine B |

It has been found that Pseudomonas aeruginosa can be identified as the microorganism when analytes are detected in sections A1, A2, A3, B1 and C3. Similarly, Staphylococcus aureus can be identified when sections A2, B1, B3, C1, C2 and C3 contain analyte captured thereon. In this case, capture of an analyte in section C1 is sufficient for taxonomic identification. Capture of cells in sections A2, B1, B3, C2 and C3 reinforces the result. The incorporation of multiple ligands targeting a given analyte onto a sensor chip, in effect, permits multiple, independent analyses to be carried out using a single sample. This increases the statistical reliability of the analytical outcome.

The various ligands are preferably tethered to a substrate by means of organic coupling agents which are themselves well known to those skilled in the art. Glass, metal or polymer substrates are employed that exhibit chemical moieties that can be themselves modified, or that can be chemically oxidized to produce exposed hydroxyl groups or other surface-exposed functional groups for modification. Without limiting the invention as to theory, various molecules can be reacted with the surface-exposed functional group, in sequence, to produce a reactive tether that is sufficiently far from the surface to practice the invention. The ligand is then tethered to the surface of the substrate through the coupling agent (i.e., the synthesized organic linker). Further, the linker should be of sufficient length to present the ligand at the optimal distance (around the distance of 40 Ångstroms) from the surface of the chip. This observation is based on our determination that shorter distances results in decreased bacterial cell capture efficiency. The chemical reactions used in tethering ligands to the surface of the sensor chip are known to those skilled in the art and are described in the literature. Such reactions may be found in G. T. Hermanson Bioconjugate Techniques (San Diego: Academic Press, 1966); Hansson et al., “Carbohydrate-Specific Adhesion of Bacteria to Thin Layer Chromatograms: A Rationalized Approach to the Study of Host Cell Glycolipid Receptors” (Analytical Biochemistry 146: 158-163 (1985)); and, Nilsson et al., “A Carbohydrate Biosensor Surface for the Detection of Uropathogenic Bacteria” (Bio/Technology 12: 1376-1378 (December 1994)).

Illustrative of such reactions are those used to tether a NHS-ester heme derrivative as a ligand to the surface of a polyacetal polymer sensor chip. In the first stage, the polyacetal polymer surface is treated with acid to activate the surface and generating free hydroxyl groups (after a thorough water wash) which are reacted with a basic solution of chloroacetic acid to add an acetic acid ester to the surface:
Polymer Surface-OH+Cl—CH₂CO₂H→Polymer Surface-O—CH₂CO₂H
The product of that reaction can then be reacted with 1-ethyl-3-(3-dimethylaminopropyl)carbodiimide hydrochloride at a pH between 4.7 and 6.0 in the presence of N-hydroxysuccinidide (NHS) to form the corresponding o-acylisourea intermediate:
Polymer Surface-O—CH₂CO₂—C(NH—C₂H₅)═N—(CH₂)₃—N⁺(CH₃)₂H
This somewhat labile o-acylisourea intermediate reacts spontaneously with the NHS present in the solution to yield the following stable NHS-ester:
Polymer Surface-O—CH₂C(O)—NHS
The NHS-ester, in turn, can be reacted with 1,6-diaminohexane to yield:
Polymer Surface-O—CH₂C(O)—NH—(CH₂)₆—NH₂
Next, the product of the preceding reaction is reacted with disuccinimidyl suberate yielding the terminal NHS ester:
Polymer Surface-O—CH₂C(O)—NH—(CH₂)₆—NH—C(O)—(CH₂)₆—C(O)—NHS
which can then be reacted with 1,12-diaminododecane to yield:
Polymer Surface-O—CH₂C(O)—NH—(CH₂)₆—NH—C(O)—(CH₂)₆—C(O)—NH—(CH₂)₁₂—NH₂

The foregoing surface-attached tether can then be reacted with the NHS— derivative of heme (shown below) to yield a covalently tethered heme ligand which is at around 40 Å from the surface.

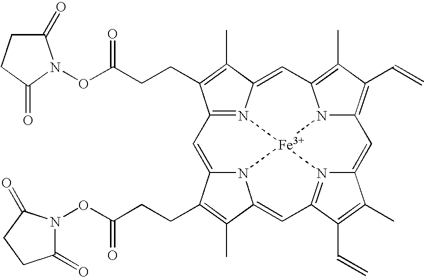

It will be understood that various changes and modifications can be made in the determination, procedure, and formulation without departing from the spirit of the invention, especially as defined in the following claims:

What is claimed:

1. A method for the rapid identification of a biological analyte comprising:
a. exposing a solution containing the analyte to a ligand specific for the analyte of interest that has been covalently bound directly to a photostable linker, said linker covalently tethered to a substrate surface wherein said photostable linker has a length of between 35 Å and 50 Å;
b. separating the bound analyte from the non-binding components of the solution containing the analyte by physical separation of the substrate surface from the sample, washing or both; and
c. interrogation of the ligand-tethered substrate surface for analyte binding with a detection method capable of detecting the bound analyte, whereby identification of the analyte is provided through detection of binding between the analyte and the specific substrate-tethered ligand.

2. The method of claim 1, wherein the biological analyte is selected from the group comprised of:
a. proteinaceous toxins;
b. cytosolic proteins, wherein said protein is obtained from the microorganism by exposing the microorganism-containing sample to conditions that result in the rupture of said microorganism and the spilling of said microorganism's contents into a solution, wherein said exposure treatments include:
i. proteinaceous toxins;
ii. holins;
iii. enzymatic treatment;
iv. plasma discharge;
v. freeze-thaw cycling;
vi. sonication; and
vii. bacteriophage infection
c. proteinaceous material of diagnostic utility.

3. The method of claim 1, wherein the ligand is a peptide, comprised of three to twenty amino acids, specific for a proteinaceous toxin.

4. The method of claim 1, wherein the ligand is a peptide, comprised of three to twenty amino acids, specific for a proteinaceous hormone.

5. The method of claim 1, wherein the ligand is a peptide, comprised of three to twenty amino acids, specific for a cytosolic protein.

6. The method of claim 1, wherein the ligand is a peptide, comprised of three to twenty amino acids, specific for a protein with diagnostic utility.

7. The method of claim 1, wherein the ligand is a peptide that does not contain tryptophan or tyrosine and detection of the captured analyte is accomplished through interrogation of the surface to detect an intrinsic fluorescence of the tryptophan and/or tyrosine residues present in the captured protein where said intrinsic fluorescence is detected between 300 and 400 nm upon excitation by ultraviolet light between 200 and 300 nm.

8. The method of claim 1, wherein the detection of the captured analyte is accomplished through the fluorescence of a reactive dye conjugate exposed to the protein before capture of the analyte by the tethered ligand surface.

9. The method of claim 1, wherein the detection of the captured analyte is accomplished through interrogation of the surface to detect the fluorescence of a reactive dye conjugate exposed to the protein after capture of the analyte by the tethered ligand surface.

10. The method of claim 1, wherein the detection of the captured analyte is accomplished through interrogation of the surface to detect the radioactivity of a reactive compound exposed to the protein before capture of the analyte by the tethered ligand surface.

11. The method of claim 1, wherein the detection of the captured analyte is accomplished through interrogation of the surface to detect the radioactivity of a reactive compound exposed to the protein after capture by the tethered ligand surface.

12. The method of claim 1, wherein the detection of the captured analyte is accomplished through interrogation of the surface to detect the luminescence of a reactive dye conjugate exposed to the protein before capture of the analyte by the tethered ligand surface.

13. The method of claim 1, wherein the detection of the captured analyte is accomplished through interrogation of the surface to detect the luminescence of a reactive dye conjugate exposed to the protein after capture of the analyte by the tethered ligand surface.

14. The method of claim 1, wherein the detection of the captured analyte is accomplished through interrogation of the surface to detect the phosphorescence of a reactive dye conjugate exposed to the protein before capture of the analyte by the tethered ligand surface.

15. The method of claim 1, wherein the detection of the captured analyte is accomplished through interrogation of the surface to detect the phosphorescence of a reactive dye conjugate exposed to the protein after capture of the analyte by the tethered ligand surface.

16. The method of claim 1, wherein the detection of the captured analyte is accomplished through interrogation of the surface to detect the optical absorbance of a reactive dye conjugate exposed to the protein before capture of the analyte by the tethered ligand surface.

17. The method of claim 1, wherein the detection of the captured analyte is accomplished through interrogation of the surface to detect the optical absorbance of a reactive dye conjugate exposed to the sample after capture of the analyte by the tethered ligand surface.

18. The method of claim 1, wherein the detection of the captured analyte is accomplished through interrogation of the surface to detect the fluorescent quenching of the fluorescent tethered ligand surface upon binding of the protein.

19. A method for identification of a protein analyte comprising:
a. exposing a solution containing the protein analyte to an array of different peptide ligands which have been covalently tethered with a photostabile linker to a substrate surface at a distance between 35 and 50 Å from the substrate surface;
b. separating the bound protein analyte on the ligand array from the non-binding components of the solution by physical separation of the substrate surface from the sample, washing or both; and
c. interrogating the ligand-tethered substrate surface with a detection method capable of detecting the bound analyte for protein analyte binding through the:
i. intrinsic fluorescence of the tryptophan and/or tyrosine residues present in the captured protein where said intrinsic fluorescence is detected between 300 and 400 nm upon excitation by ultraviolet light between 200 and 300 nm;
ii. fluorescence of a reactive dye conjugate exposed to the protein after capture of the analyte by the tethered ligand surface;
iii. radioactivity of a reactive compound exposed to the protein after capture by the tethered ligand surface;
iv. luminescence of a reactive dye conjugate exposed to the protein after capture of the analyte by the tethered ligand surface;
v. phosphorescence of a reactive dye conjugate exposed to the protein after capture of the analyte by the tethered ligand surface;
vi. optical absorbance of a reactive dye conjugate exposed to the sample after capture of the analyte by the tethered ligand surface;
vii. the fluorescent quenching of the fluorescent tethered ligand surface upon binding of the protein.
d. wherein the protein analyte is:
i. a proteinaceous toxin
ii. a cytosolic protein; and
iii. a proteinaceous hormone.

20. A method for the rapid identification of a biological analyte comprising:
a. exposing a solution containing the analyte to a ligand specific for the analyte of interest, said analyte of interest having been first conjugated to a marker, said ligand having been covalently bound directly to a photostable linker, said linker covalently tethered to a substrate surface wherein said photostable linker has a length of between 35 Å and 50 Å;
b. separating the bound analyte from the excess marker-conjugated ligands, wherein said separation occurs through:
i. separating the bound analyte from the non-binding components of the solution containing the analyte by physical separation of the substrate surface from the sample, washing or both;
ii. chromatography, wherein the stationary phase of the column contains the covalently tethered ligands, said ligands being bound to the stationary phase surfaces via photostable linkers; and
iii. magnetic separation, wherein the ligand is conjugated to a magnetic particle and the separation of the bound analyte from the non-binding components of the analyte solution is accomplished by magnetic separation with the ligand being tethered to the magnetic particle.
c. interrogation of the ligand-tethered substrate surface for analyte binding with a detection method capable of detecting the bound analyte, whereby identification of the analyte is provided through detection of binding between the analyte and the specific substrate-tethered ligand.

21. The method of claim 20, wherein the biological analyte is selected from the group comprised of:
a. bacteria;
b. viruses;
C. proteinaceous toxin;
d. rickettsiae;
e. protozoa;
f. fungi;
g. cytosolic protein; and
h. proteinaceous material of diagnostic utility.

22. The method of claim 20, wherein the ligand is selected from the group containing:.
a. heme compounds;
b. siderophores;
c. polysaccharides;
d. peptides specific for outer membrane proteins; and
e. peptides specific for conjugated lipids.

23. The method of claim 20, wherein the marker is fluorescent and the detection is via fluorescence.

24. The method of claim 20, wherein the marker is luminescent and the detection is via luminescence.

25. The method of claim 20, wherein the marker is radioactive and the detection is via radioactivity.

26. The method of claim 20, wherein the marker is phosphorescent and the detection is via phosphorescence.

27. A method for capture of a biological analyte from a fluid onto a substrate whereby the sample is passed over a substrate surface that has been conjugated with non-antibody ligands through photostable tethers, said tethers having a length of between 35 Å and 50 Å, wherein:
a. the ligands used are selected from the group consisting of: heme compounds, siderophores, polysaccharides, and peptides specific for outer membrane proteins, conjugated lipids, prions, and microbial protein targets;
b. the substrate is suitable for a chromatographic stationary phase;
c. the biological analytes are selected from the groups of: bacteria, viruses, rickettsiae, protozoa, fungi, prions, microbial protein targets, and proteinaceous matter of diagnostic utility;
d. the fluid from which the biological analytes are captured on the substrate surface via the tethered ligands are from samples selected from the group consisting of:
i. water samples;
ii. medical samples;
iii. veterinary samples;
iv. aerosol samples;
V. food product slurries;
vi. food ingredient slurries; and
vii. soil slurries.


SEQUENCE LISTING (3 sequences)

SEQ ID NO: 1 - protein, 11 aa, Staphylococcus aureus
  PEPTIDE (1)..(11): Artificial peptide that binds to Protein A (membrane protein) of Staphylococcus aureus
GHHKHHHGGGC

SEQ ID NO: 2 - protein, 22 aa, Staphylococcus aureus
  PEPTIDE (1)..(22): Artificial peptide that binds to toxic shock syndrome toxin-1 protein from Staphylococcus aureus
GADRSYLSFIHLYPELAGAGGC

SEQ ID NO: 3 - protein, 11 aa, Staphylococcus aureus
  PEPTIDE (1)..(6): Amino acid sequence residues 1 to 6 of Clone 14 in Table I. GGGGC added for spacers and way of conjugating peptide to tether.
WHKAPRGGGGC